(19) United States
(12) Patent Application Publication
(10) Pub. No.: US 2012/0116515 A1
(43) Pub. Date: May 10, 2012

(54) DEMINERALIZED CORTICAL BONE IMPLANTS

(76) Inventors: Eric J. Semler, Piscataway, NJ (US); Clint Boylan, Minneapolis, MN (US); Karen Roche, Stillwater, MN (US)

(21) Appl. No.: 13/277,703

(22) Filed: Oct. 20, 2011

Related U.S. Application Data
(60) Provisional application No. 61/406,283, filed on Oct. 25, 2010.

(51) Int. Cl. A61F 2/44; A61F 2/28

(52) U.S. Cl. 623/17.16; 623/23.63

(57) ABSTRACT

Implants comprising a plurality of separate cortical bone units, which have been at least partially demineralized and are osteoinductive, are described herein. The implants can be used in methods for treating bone. Also, disclosed are methods for treating spinal conditions using these implants. The spinal conditions include but are not limited to repairing damage to or defects in the spine, such as fractures in a vertebral body or degeneration of spinal discs.

### FIELD OF THE INVENTION

Implants comprising a plurality of separate cortical bone units, which have been at least partially demineralized and are osteoinductive, are described herein. The implants can be used in methods for treating bone. Also disclosed are methods for treating spinal conditions using these implants. The spinal conditions include but are not limited to repairing damage to or defects in the spine, such as fractures in a vertebral body of a patient or degeneration of a spinal disc in a patient.

### BACKGROUND

Fractures, such as compression or burst fractures, in the vertebral bodies of the spine are common in elderly patients who suffer from osteoporosis. There are approximately 700,000 cases of pathologic vertebral body compression fractures reported annually in the United States. As the patient's bone weakens, the vertebral bodies lose height and collapse, leading to severe pain and deformity. Burst and compression fractures of the vertebral bodies also occur in trauma cases, again leading to pain and deformities.

To treat fractures in vertebral bodies, bone cement (e.g., PMMA) is often used either in procedures that involve direct injection of the bone cement into the fractured vertebral body (i.e., vertebroplasty) or injection of the bone cement into the vertebral body after the height of the vertebral body is restored using a pressurized balloon (i.e., kyphoplasty). One of the disadvantages of using bone cement is that, once it is injected inside the patient, the bone cement is an inorganic material that acts as a foreign body, and thus, does not allow for complete healing and may instead lead to bone disease. Moreover, bone cement is typically stiffer than bone, which may increase the incidence of adjacent level fractures in the spine. Also, bone cement leakage may cause complications, and has been reported to occur in vetebroplasty and kyphoplasty procedures. If leakage does occur, PMMA bone cements can cause soft tissue injury due to the high temperatures of the exothermic polymerization reaction. In addition, PMMA forced into the vascular system can cause emboli.

Accordingly, minimally invasive approaches to repairing fractured vertebral bodies are desirable. Also a bone material for repairing the fracture that does not leak, and allows for easy handling and delivery, as well as complete healing post-implantation, is also desirable.

Another common spinal condition is mild to severe degenerative disc disease. A healthy intervertebral disc facilitates motion between pairs of vertebrae while absorbing and distributing compression forces and torque forces. The disc is composed of two parts; namely a tough outer ring (the annulus fibrosis (AF)) which holds and stabilizes a soft central core material (the nucleus pulposus (NP)) that bears the majority of the load forces. With degenerative disc disease, the onset of the degenerative cascade in the intervertebral disc(s) is typically associated with dehydration and loss of volume of the NP. The NP may then leak or bulge into the AF, and either or both of the NP and AF may come into contact with spinal nerves. This can cause inflammation or micromotion instability, resulting in pain, and loss of motion.

When an intervertebral disc is deformed, ruptured, diseased, or degenerating, surgical treatment can consist of augmenting or repairing the disc. For example, materials may be implanted or injected into the disc to replace or augment the NP. Also, to treat intervertebral discs, surgical treatments have been used to create a fusion between the two adjacent vertebral bodies. Prior approaches to vertebral fusion have involved substantial invasive surgery. It would be advantageous to have a vertebral fusion using an implant that is minimally invasive. In order to achieve a successful minimally invasive delivery of the implant into the disc space for fusion, the implant material must be able to easily pass through a small diameter cannula into the surgical site without jamming or wedging. Moreover, it is desirable to have a maximal amount of surface area onto which new bone can begin to grow or form. In addition, certain materials that have been used do not allow bone healing through the entire implant to achieve complete interbody fusion since they may be synthetic materials that do not remodel into bone. Additionally, while implants have been used for spinal fusion, it has not always been possible to size an implant to fit the implant site. Also, the implants have not necessarily had the ability to conform to the shape of the implant site such that the contact between the implant and the endplates of the vertebral bodies is maximized. Moreover, the materials that have been used for vertebral fusion, such as titanium and polyether-etherketone (PEEK), do not always provide the optimal degree of mechanical support. Also, implant materials that are radiopaque do not allow for newly formed bone to be readily detected during follow-up x-rays.

Accordingly, minimally invasive approaches, in which the implant materials do not jam or wedge during extrusion from delivery tubes or containers to implant sites, are desirable. Also desirable are implant materials that promote bone growth or healing, can be sized and can conform to the shape of the implant site, provide adequate mechanical support/load bearing and/or are at least partially radiolucent.

### SUMMARY OF THE INVENTION

The implants described herein comprise a plurality of separate cortical bone units that are at least partially demineralized. The cortical bone units are osteoinductive. The cortical bone units can have at least one dimension greater than about 1.0 mm. When the cortical bone units are implanted into a cavity that has a volume, there are void spaces between the cortical bone units in the cavity. In certain embodiments, the cavity is located in a patient. Also, in some embodiments, the cortical bone units can be implanted into an implantable container, (e.g., an expandable, porous container), located within the cavity. The implantable container can be a mesh bag. The cortical bone units can have a cylindrical, spherical, pyramidal, ovoid, discoid, oblong, or cuboidal shape. Also, in some embodiments, the cortical bone units can have at least one dimension from about 1.5 mm to about 5.0 mm, from about 2.0 mm to about 3.0 mm, or greater than or equal to about 2.5 mm.

In certain embodiments, the implants can be free of cortical bone units having at least one dimension less than about 1.0 mm, or include about 5% by weight or less, or about 1% to about 5% by weight of cortical bone units having at least one dimension less than about 1.0 mm. Also, in certain embodiments, the implants can be free of cortical bone units where all the dimensions are less than about 1.0 mm, or include about 5% by weight or less, or about 1% to about 5% by weight of cortical bone units where all the dimensions are less than about 1.0 mm. In addition, the cortical bone units of the implants can be derived from allograft bone. Furthermore, the implants can be free of cancellous bone, or include about 1% by weight or less, or about 1% to about 5% by weight of cancellous bone.

Moreover, the implants can include cortical bone units that are at least partially or fully demineralized. In some embodiments, the implants can be free of non-demineralized bone or include less than or equal to about 1% by weight, or about 1% to about 5% by weight of non-demineralized bone. Preferably, the cortical bone units are demineralized to have a calcium content of less than or equal about 0.5% wt. At this level of demineralization, the cortical bone units of the implants will be radiolucent prior to or during implantation of the implant into a patient. The implants will remain radiolucent in the patient until new calcified bone has begun to form at the surgical site. In some embodiments, the implants remain radiolucent for up to about 15 weeks or up to about 6 months. In certain embodiments, the implants remain radiolucent for about 2 weeks to about 6 months, for about 6 weeks to about 24 weeks, or for about 6 weeks to about 12 weeks. In certain embodiments, the implants can comprise a radiopaque marker.

Furthermore, the implants described herein can also comprise a carrier. In certain embodiments, the carrier can comprise saline, sodium hyaluronate or hyaluronic acid. The carrier can be mixed with the cortical bone units. In other embodiments, the implants can be free of a carrier, or includes less than or equal to about 1% by weight or about 1% to about 5% by weight of a carrier. In yet other embodiments, the carrier can comprise a lubricant. Alternatively, the carrier can be free of a lubricant, or includes less than or equal to about 1% by weight or about 1% to about 5% by weight of a lubricant.

In addition, in certain embodiments, when the cortical bone units of the implants are implanted, the cortical bone units can occupy about 75% to about 99%, or about 80% to about 90% of the volume of the cavity or implantable container. Also, when the cortical bone units of the implants are implanted the packing density of the cortical bone units in the cavity can be about 0.5 g/cc to about 1.0 g/cc, or about 0.6 glee to about 0.8 g/cc based on the dry weight of the bone units or implant material.

Moreover, described herein are methods of treating bone, including but not limited to spinal vertebrae. The methods comprise forming at least one cavity, having a volume and at least one opening, within the bone. The methods further comprise implanting into the cavity an implant as described herein. For example the implant can comprise a plurality of separate cortical bone units that are at least partially demineralized and osteoinductive. The cortical bone units can have at least one dimension greater than about 1.0 mm. Also, after the implant has been implanted in the cavity there can be void spaces between the cortical bone units in the cavity.

In some embodiments, the methods further comprise sealing the opening of the cavity after the implant has been implanted in the cavity. The opening can be sealed with a biocompatible sealant, such as an allograft bone plug, a ceramic plug, polymeric plug, metallic plug, or a fibrin glue. Also, the methods can further comprise inserting an implantable container into the cavity prior to implanting the implant into the cavity so that when the implant is implanted into the cavity, the implant will be contained in the implantable container. The implantable container can be expandable and/or porous to allow for bone formation between the surrounding bone and the implant material. In certain embodiments, the container can be a mesh bag. In addition, when the implant is contained in the implantable container, the implant can have a volume that is greater than the volume of the cavity.

Moreover, prior to implanting the implant in the cavity, the plurality of cortical bone units can be packaged in a delivery container, such as a cannula, syringe, cartridge, hollow rod, hollow delivery tube, or fill tube. The plurality of cortical bone units can be situated in a single row in the delivery container. The methods can comprise dispensing one cortical bone unit or multiple cortical bone units at a time from the delivery container into the cavity.

Also described herein are methods of treating a vertebral body in a patient, such as methods of treating fractured vertebral bodies. The methods comprise forming at least one cavity, having a volume and at least one opening, within the vertebral body. The methods further comprise implanting into the cavity an implant as described herein, e.g., the implant can comprise a plurality of separate cortical bone units that are at least partially demineralized and osteoinductive, and the cortical bone units can have at least one dimension greater than about 1.0 mm. Also, after the implant has been implanted in the cavity of the vertebral body there can be void spaces between the cortical bone units in the cavity.

In some embodiments, the methods of treating a vertebral body further comprise sealing the opening of the cavity after the implant has been implanted in the cavity as described above. The opening can be sealed with a biocompatible sealant. Also, the methods can further comprise inserting an implantable container into the cavity prior to implanting the implant into the cavity so that when the implant is implanted into the cavity, the implant will be contained in the implantable container. The implantable container can be expandable and/or porous to allow for bone formation between the surrounding bone and the implant material. The container can be a mesh bag. In addition, when the implant is contained in the implantable container, the implant can have a volume that is greater than the volume of the cavity.

Moreover, prior to implanting the implant in the cavity, the plurality of cortical bone units can be packaged in a delivery container, such as a cannula, syringe, cartridge, hollow rod, hollow delivery tube, or fill tube. The plurality of cortical bone units can be situated in a single row in the delivery container. The methods can comprise dispensing one cortical bone unit or multiple cortical bone units at a time from the delivery container into the cavity.

Additionally, described herein are methods of treating a spinal disc in a patient, such as methods for treating degenerated spinal discs in the patient. The methods comprise forming at least one cavity, between two adjacent vertebral bodies, wherein the cavity has a volume and at least one opening into the cavity. In some embodiments, the cavity can be located in the spinal disc. The methods further comprise implanting into the cavity an implant as described herein. After the implant has been implanted in the cavity there are void spaces between the cortical bone units in the cavity. In some embodiments, the implant can be used to create a fusion between the two adjacent vertebral bodies.

Furthermore, in some embodiments, at least one of the vertebral bodies has an endplate and the methods can further comprise decorticating the endplate prior to implanting the implant into the cavity. Also, the methods can comprise the step of sealing the opening of the cavity after the implant has been implanted into the cavity with a biocompatible sealant such as an allograft bone plug, a ceramic plug, polymeric plug, metallic plug, or a fibrin glue.

As with the methods for treating a vertebral body, the methods for treating a spinal disc can further comprise the step of inserting an implantable container, such as an expandable and/or porous container, into the cavity prior to implanting the implant into the cavity so that when the implant is implanted into the cavity, the implant will be contained in the implantable container. The container can be a mesh bag. Also, when the implant is contained in the implantable container, the implant can have a volume that is greater than the volume of the cavity. In addition, prior to insertion of the implant in the cavity, the plurality of cortical bone units can be contained in a delivery container, such as a cannula, syringe, cartridge, hollow rod, hollow delivery tube, or fill tube. The plurality of cortical bone units can be situated in a single row in the delivery container. The methods can further comprise dispensing one cortical bone unit or multiple bone units at a time from the delivery container into the cavity.

The implants and methods of treatment described herein provide certain advantages. One advantage is that the implants comprise a plurality of separate cortical bone units, which are harder, firmer, and denser than other materials, such as spongy, cancellous bone or bone powders, which are used in other spinal implants. Thus, the implants described herein are well suited to load bearing applications when inserted inside a cavity. When the implants are used in a spinal application, they may be used to stabilize the surrounding vertebrae after implantation.

An additional advantage of the implants described herein is that the cortical bone units are relatively large, e.g., having at least one dimension greater than about 1.0 mm, compared to other materials used for treating spine conditions. Because of their size, when the cortical bone units are inserted or packed into a cavity or implantable container located within a cavity in a patient, there are void spaces between the cortical bone units, i.e., the cortical bone units occupy less than 100% of the volume of the cavity or container. These void spaces help promote healing of the surrounding bone by providing channels for blood and growth factors to pass through and create more surface area for cell attachment and remodeling.

Furthermore, compared to powdered materials, such as bone powders, for treating spinal conditions, the implants comprising cortical bone units described herein can be easier for surgeons or other medical personnel to insert into a patient during the time of minimally invasive spine surgery through a small diameter cannula. Powdered materials that are used to treat spinal conditions are often placed in tubes or containers. The surgeons or medical personnel deliver the powdered material to the patient by extruding, the powdered material from the tube or container. Because the powdered material has relatively small particle sizes, the material has a tendency to become packed and forms a dense mass in the tube or container. The packing of the powdered material in the tube or container can cause a jam therein that makes it difficult for the surgeon to extrude the powdered material. In contrast, because of the relatively larger size of the cortical bone units described herein, the cortical bone units do not form a dense mass that can jam the tube or container from which they are being delivered. In certain embodiments, the cortical bone units can be delivered from the tube or container in which they are placed, so that they are dispensed in single file from tubes or containers. The size of the cortical bone units may also eliminate the need for a carrier, which may be needed when using powdered material where the carrier is included to create flowability and avoid or reduce the jamming problems that occur when using powdered material.

As discussed above, because of the relatively large size of the cortical bone units, when the cortical bone units are placed into a cavity or container located within a cavity in a patient, there are void spaces between the individual cortical bone units. Due to these void spaces, and despite the greater density of cortical bone compared to cancellous bone, the overall packing density of the implants described herein has been found to be lower than the packing density of similar implants comprising a mixture of smaller particles of cortical and cancellous bone mixed with cortical bone powder, i.e. corticocancellous implants. Surprisingly, despite their lower packing density, the implants described herein have been found to have the same or better capacity for load bearing than the corticocancellous implants.

Another advantage of the implants described herein is that the cortical bone units of the implant are demineralized such that the cortical bone units are radiolucent before or during insertion of the implant. More specifically, it is often desirable to be able to visualize the formation of new bone at the implantation site over time with standard radiographic imaging techniques. Since the implant material is demineralized, it will appear radiolucent until new calcified bone appears. If non-demineralized bone were to be used in the implant, the implant would appear radiopaque at the time of surgery and it would be difficult to discern the implant material from the surrounding mineralized bone. Therefore, the physician will not be able to easily differentiate between the formation of new bone and the bone that was used to make the implant. Indeed, the implants described herein may be radiolucent for up to about 15 weeks, or for up to about 6 months as the implant material begins to remodel and new bone begins to form. In some embodiments, the implants may be radiolucent for about 2 weeks to about 6 months, for about 6 weeks to about 24 weeks, or for about 6 weeks to about 12 weeks.

### BRIEF DESCRIPTION OF THE DRAWINGS

FIGS. 1A and 1B a perspective view of a plurality of cortical bone units, that are box, cube, cylinder, disc, sphere or pyramid shaped. FIG. 1B shows the cortical bone units contained in a fill tube container. The cortical bone units are situated in a single row in the fill tube container so that the cortical bone units can be dispensed from the fill tube container a single-file order.

FIG. 2 shows a cavity within a vertebral body having a compression or burst fracture. An implantable container, which has been placed within the cavity, is being filled with cortical bone units from a delivery container.

FIG. 3 shows a cavity located between two adjacent vertebral bodies. An implantable container, which has been placed within the cavity, is being filled with cortical bone units from a delivery container.

FIG. 4 shows a comparison between the packing density of a test sample comprising demineralized cortical bone units rehydrated with saline and the packing density of a control sample of demineralized cortical powder, corticocancellous granules, and sodium hyaluronate carrier, when the samples are exposed to certain sustained applied pressures.

FIG. 5A shows a histology sample of an unfilled or empty void in a sheep vertebral body 6 weeks after the void was created. FIG. 5B shows a radiograph of the vertebral body shown in FIG. 5A.

FIG. 6A shows a histology sample of an unfilled or empty void in a sheep vertebral body 12 weeks after the void was created. FIG. 6B shows a radiograph of the vertebral body shown in FIG. 6A.

FIG. 7A shows a histology sample of a void in a sheep vertebral body 6 weeks after the void was filled with a test composition comprising demineralized cortical bone units and sodium hyaluronate. FIG. 7B shows a radiograph of the vertebral body shown in FIG. 7A.

FIG. 8A shows a histology sample of a void in a sheep vertebral body 12 weeks after the void was filled with a test composition comprising demineralized cortical bone units and sodium hyaluronate. FIG. 8B shows a radiograph of the vertebral body shown in FIG. 8A.

FIG. 9A shows a histology sample of a void in a sheep vertebral body 6 weeks after the void was filled with a test composition comprising demineralized cortical bone units and phosphate buffered saline. FIG. 9B shows a radiograph of the vertebral body shown in FIG. 9A.

FIG. 10A shows a histology sample of a void in a sheep vertebral body 12 weeks after the void was filled with a test composition comprising demineralized cortical bone units and phosphate buffered saline. FIG. 10B shows a radiograph of the vertebral body shown in FIG. 10A.

FIG. 11A shows a histology sample of a void in a sheep vertebral body 6 weeks after the void was filled with a control composition comprising non-demineralized corticocancellous granules, demineralized cortical bone powder and sodium hyaluronate. FIG. 11B shows a radiograph of the vertebral body shown in FIG. 11A.

FIG. 12A shows a histology sample of a void in a sheep vertebral body 12 weeks after the void was filled with a control composition comprising non-demineralized corticocancellous granules, demineralized cortical bone powder and sodium hyaluronate. FIG. 12B shows a radiograph of the vertebral body shown in FIG. 12A.

### DETAILED DESCRIPTION OF THE INVENTION

### Definitions

As used herein, and unless otherwise defined, the term “separate cortical bone unit” or “cortical bone unit” refers to a unit of bone that is made of cortical bone and that is not connected to another bone unit.

As used herein, and unless otherwise defined, the term “osteoinductivity” or “osteoinductive” refers to a material's ability to lead to the formation of new bone.

As used herein, and unless otherwise defined, the term “osteoconductivity” or “osteoconductive” refers to a material's ability to provide a suitable structure or scaffold for the growth of new bone.

As used herein, and unless otherwise defined, the term “at least partially demineralized,” when used in connection with bone, refers to bone that has had at least a portion of its calcium content removed.

As used herein, and unless otherwise defined, the term “fully demineralized,” when used in connection with bone, refers to bone that has had calcium removed from the bone so that the residual calcium content is less than or equal to about 0.5 weight percent of the bone.

As used herein, and unless otherwise defined, the term “non-demineralized,” when used in connection with bone, refers to bone that has not had calcium removed from the bone.

As used herein, and unless otherwise defined, the term “radiolucent” refers to a material, such as bone, that cannot be visualized with radiological techniques.

As used herein, and unless otherwise defined, the term “radiopaque” refers to a material, such as bone, that can be visualized with radiological techniques.

As used herein, and unless otherwise defined, the term “void spaces between the cortical bone units” refers to spaces between cortical bone units that are not occupied by cortical bone units or any other solid material.

As used herein, and unless otherwise defined, the term “packing density of the cortical bone units” in a container or cavity refers to the dry mass of cortical bone units present in the container or cavity per unit volume of the container or cavity.

### Cortical Bone Units

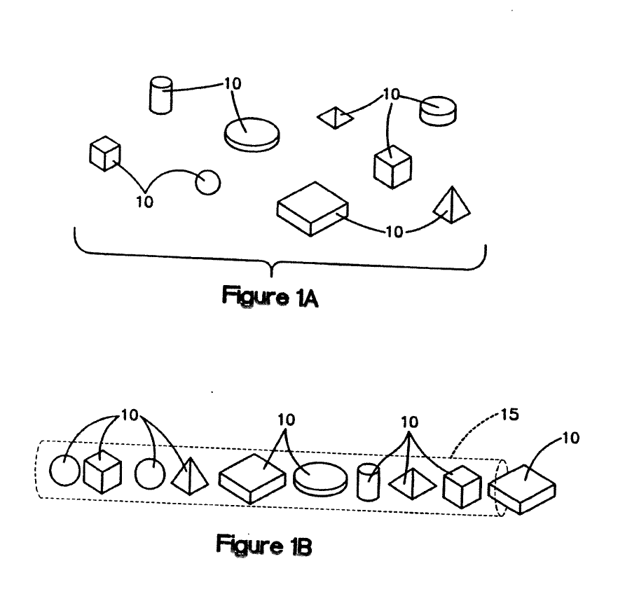

The implants described herein comprise a plurality of separate cortical bone units. FIG. 1A shows a plurality of separate cortical bone units 10. As shown in this figure, the cortical bone units 10 are discrete or not connected to each other. FIG. 1B shows a plurality of the cortical bone units 10 contained in a delivery container 15, such as a delivery tube or cannula, which can facilitate the delivery and implantation of the cortical bone units 10 to a patient. In this embodiment, the cortical bone units 10 are situated in a single row in the delivery container 15. In other embodiments, the cortical bone units can be situated in the delivery container in different arrangements.

As shown in FIG. 1A, the cortical bone units 10 can have a variety of geometric shapes. For example, the cortical bone units may have a particular shape, including, but not limited to, a cylindrical, spherical, pyramidal, ovoid, discoid, oblong (i.e., box) or cuboidal shape. In addition, the implant can comprise cortical bone units having the same shape or a variety of shapes. For instance, at least one of the cortical bone units can have a particular shape, while other cortical bone units have a different shape.

In some embodiments, the cortical bone units have at least one dimension from about 0.5 mm to about 10 mm, about 0.75 mm to about 9 mm, about 0.85 mm to about 8 mm, 1.0 mm to about 10 mm, about 1.0 mm to about 9 mm, about 1.0 mm to about 8 mm, about 1.0 mm to about 7 mm, about 1.0 mm to about 6 mm, about 1.5 mm to about 5 mm, about 1.5 mm to about 4 mm, about 1.5 mm to about 3 mm, or about 2 mm to about 3 mm. In particular embodiments, the at least one dimension may be the height, width, length, thickness and/or diameter of the cortical bone unit.

In addition, in some embodiments, the cortical bone units may have at least one dimension that is greater than or equal to about 0.1 mm, about 0.25 mm, about 0.5 mm, about 0.75 mm, about 0.8 mm, about 0.85 mm, about 0.9 mm, about 0.95 mm, about 1.0 mm, about 1.25 mm, about 1.5 mm, about 1.75 mm, about 2.0 mm, about 2.25 mm, about 2.5 mm, about 2.75 mm, about 3.0 mm, about 3.25, about 3.5 mm, about 3.75 mm, about 4.0 mm, about 4.25 mm, about 4.5 mm, about 4.75 mm, about 5.0 mm, about 5.5 mm, about 6.0 mm, about 6.5 mm, about 7.0 mm, about 7.5 mm, about 8.0 mm, about 8.5 mm, about 9.0 mm, about 9.5 mm or about 10.0 mm.

In certain embodiments, the cortical bone units have an oblong shape and comprise a first dimension and a second dimension. The first and second dimension may each be about 1 mm to about 3 mm.

Furthermore, the implants may be free of or contain less than or equal to about a certain percent by weight, such as about 0.5% to about 25% by weight, of cortical bone units having at least one or more dimensions less than or equal to about 0.1 mm, about 0.2 mm, about 0.3 mm, about 0.4 mm, about 0.5 mm, about 0.6 mm, about 0.7 mm, about 0.8 mm, about 0.9 mm, or about 1.0 mm. In some embodiments, the implant may contain about 0.5% to about 25% by weight, about 1% to about 10% by weight, or about 1% to about 5% by weight of cortical bone units having at least one or more dimensions less than the above dimensions. In certain embodiments, the implants may contain less than or equal to about 0.5% by weight, about 1.0% by weight, about 5.0% by weight, about 10.0% by weight, about 15.0% by weight, about 20.0% by weight or about 25.0% by weight of cortical bone units having at least one or more dimensions less than the above dimensions.

Also, the cortical bone units may be derived from autograft bone, allograft bone, or xenograft bone. In particular embodiments, the cortical bone units are derived from allograft bone. In some embodiments, the cortical bone units are derived from a mammal, such as a human. The cortical bone used to make the cortical bone units may be derived from any bone, including, but not limited to, the femur, tibia, humerus, fibula, radius, and ulna.

Moreover, in some embodiments, the implants may comprise cortical bone units that are made exclusively or primarily of cortical bone. The implant can comprise cortical bone in an amount greater than or equal to about 5% by weight, about 10% by weight, about 15% by weight, about 20% by weight, about 25% by weight, about 30% by weight, about 35% by weight, about 40% by weight, about 45% by weight, about 50% by weight, about 55% by weight, about 60% by weight, about 65% by weight, about 70% by weight, about 75% by weight, about 80% by weight, about 85% by weight, about 90% by weight, about 95% by weight, or about 100% by weight. In some embodiments, the implant can comprise cortical bone in an amount about 50% to about 100% by weight, about 75% to about 100% by weight, or about 85% to about 100% by weight.

Also, the implants described herein may be free of cancellous bone or substantially free of cancellous bone. The implants can comprise cancellous bone in an amount less than or equal to about 0.1% by weight, about 0.25% by weight, 0.5% by weight, about 1% by weight, about 5% by weight, about 10% by weight, about 15% by weight, about 20% by weight, about 25% by weight, about 30% by weight, about 35% by weight, about 40% by weight, about 45% by weight, about 50% by weight, about 55% by weight, about 60% by weight, about 65% by weight, about 70% by weight, about 75% by weight, about 80% by weight, about 85% by weight, about 90% by weight, about 95% by weight, or about 100% by weight. In certain embodiments, the implants can comprise cancellous bone in an amount of 0% to about 50% by weight, 0% to about 25% by weight, or 0% to about 10% by weight. Also, the cancellous bone can have dimensions like those described above in connection with the cortical bone units.

The cortical bone units described herein are preferably osteoinductive. The cortical bone units may also be osteoconductive. The osteoinductive and/or osteoconductive nature of the implants described herein may engender biological repair of a damaged vertebral body or bone with new bone formation and tissue remodeling.

The cortical bone used to prepare the cortical bone units can be cleaned to eliminate undesired substances. These undesired substances can include without limitation lipids, cells and microorganisms, e.g. viruses, bacteria. The bone can be cleaned by exposing it to a detergent or an agent that eliminates microorganisms, such as an alcohol. e.g. ethanol, or hydrogen peroxide.

Furthermore, the bone used to form the cortical bone units of the implant can be at least partially demineralized. The bone can be cleaned before and/or after it is at least partially demineralized. Also, the bone can be at least partially demineralized before or after it is milled into the cortical bone units. For example, the cross-sections of bone can be at least partially demineralized before the cross-sections are milled into the cortical bone units having the desired shape. In alternative embodiments, the bone may be milled into the cortical bone units having the desired shape prior to the demineralization process.

To at least partially demineralize the bone, the bone is placed in acid. The bone may be partially demineralized, such as surface demineralized. In some embodiments it is preferred that the bone is fully demineralized so that the bone contains less than or equal to about 0.5 weight % residual calcium. In certain embodiments, the bone can be demineralized such that it contains residual calcium in an amount less than or equal to about 0.1% by weight, about 0.2% by weight, about 0.3% by weight, about 0.4% by weight, about 0.5% by weight, about 0.6% by weight, about 0.7% by weight, about 0.8% by weight, about 0.9% by weight, about 1% by weight, about 2% by weight, about 3% by weight, about 4% by weight, about 5% by weight, about 6% by weight, about 7% by weight, about 8% by weight, about 9% by weight, about 10% by weight, about 15% by weight, about 20% by weight, about 25% by weight, about 30% by weight, about 35% by weight, about 40% by weight, about 45% by weight, about 50% by weight, about 55% by weight, about 60% weight, about 65% by weight, about 70% weight, about 75% by weight, about 80% by weight, about 85% by weight, about 90% by weight, or about 95% by weight. In some embodiments, the bone can be demineralized such that it contains residual calcium in an amount of 0% to about 25% by weight, 0% to about 10% by weight, 0% to about 5% by weight, or about 0% to about 0.5% by weight.

Also, the implants described herein may be free or substantially free of non-demineralized, i.e., mineralized bone. The cortical bone units can comprise non-demineralized bone in an amount less than or equal to about 0.1% by weight, about 0.25% by weight, about 0.5% by weight, about 1% by weight, about 5% by weight, about 10% by weight, about 15% by weight, about 20% by weight, about 25% by weight, about 30% by weight, about 35% by weight, about 40% by weight, about 45% by weight, about 50% by weight, about 55% by weight, about 60% by weight, about 65% by weight, about 70% by weight, about 75% by weight, about 80% by weight, about 85% by weight, about 90% by weight, about 95% by weight, or about 100% by weight. In some embodiments, the cortical bone units can comprise non-demineralized bone in an amount of 0% to about 25% by weight, 0% to about 10% by weight, 0% to about 5% by weight, or 0% to about 1% by weight.

In some embodiments, the demineralization causes the cortical bone units, and thus, the implant, to be radiolucent. In particular embodiments, the implant will be radiolucent and not radiopaque before or during implantation into a subject. In such embodiments, the implant will not be seen in x-rays immediately upon implantation. After time, as new bone grows, the implant site will become radiopaque and will be visible in X-rays as a way of tracking the patient's bone growth. In some embodiments, as the implant material begins to remodel and new bone begins to form, the implants described herein may be radiolucent from the time of implantation up to about 2 weeks, up to about 4 weeks, up to about 6 weeks, up to about 8 weeks, up to about 10 weeks, up to about 12 weeks, up to about 15 weeks, up to about 18 weeks, up to about 24 weeks, up to about 28 weeks, or up to about 32 weeks. In certain embodiments, the implant may be radiolucent from the time of implantation for about 2 weeks to about 6 months, for about 4 weeks to about 28 weeks, for about 6 weeks to about 24 weeks, or for about 6 weeks to about 12 weeks.

In yet other embodiments, the implants described herein may include the addition of a radiopaque marker to the cortical bone units in order to make the implant visible during surgery. The radiopaque marker may be derived from, but is not limited to, beryllium copper, brass, bronze, carbon steel, clad metals, copper, kovar, molybdenum, nickel, niobium, stainless steel, tantalum, titanium, zirconium, or other radiopaque material. Other suitable materials may include, without limitation, barium, platinum, platinum iridium, gold, and iodine-containing compounds. In a particular embodiment, the radiopaque marker may be incorporated into the implant as a separate unit in the form of a pellet or wire. In another embodiment, radiopacity may be attained by chemically binding a radiopaque marker to single or multiple cortical bone units prior to implantation. The radiopaque marker may be permanent or have a temporary lifetime. In some embodiments in which the radiopaque marker has a temporary lifetime, it has a temporary lifetime of at least one month, at least two months, at least three months, at least four months, at least five months, at least six months, or at least one year.

As seen in FIG. 1B, a plurality of cortical bone units 10 may be contained in a delivery container 15, such that the cortical bone units are capable of being dispensed from the delivery container in a single-file order. It can be advantageous for the cortical bone units to be of a size and shape that enables them to be dispensed in a single-file order. This avoids problems of the cortical bone units sliding, wedging, and jamming during delivery of the cortical bone units to the implantation site from the delivery container. The delivery container may be, without limitation, a fill tube, a syringe, a cannula, a cartridge, a hollow rod, or a hollow delivery tube. In FIG. 1B, the delivery container 15 is depicted as a fill tube. The delivery container may vary in diameter. In some embodiments, the delivery container has a diameter of about 1 mm to about 10 mm, about 1 mm to about 5 mm, or about 2 mm to about 4 mm. Furthermore, the container can be made of a radiopaque material or have at least one radiopaque marker, which would make the container visible during implantation, even though the cortical bone units are radiolucent.

In addition to cortical bone units, the implants described herein may further comprise a carrier. In embodiments having a carrier, the cortical bone units can be mixed with the carrier. Additionally, the carrier may act to preserve osteoinductivity of the cortical bone units and/or provide other biological effects, e.g., support vascularization. Also, the carrier can be used to rehydrate the cortical bone units. Therefore, in certain embodiments, the carrier may comprise a hydrating agent. In some embodiments, the cortical bone units may be suspended in the carrier. In other embodiments, the carrier may be absorbed by the cortical bone units so that surfaces of the cortical bone units are surrounded by no carrier or only small amounts of a carrier.

In some embodiments, the carrier can comprise a lubricant to reduce or eliminate any friction between the cortical bone units and the devices used to deliver the cortical bone units to an implantation site. For instance, the carrier comprising a lubricant may facilitate loading of the cortical bone unit into a delivery container, such as a fill tube, as well as delivery of the cortical bone units from the delivery container during implantation. Also, the carrier comprising a lubricant can reduce or eliminate the friction among the cortical bone units.

The carrier may be, without limitation, saline, e.g., phosphate buffered saline, or an organic carrier. Organic carriers may include, but are not limited to sodium hyaluronate, alginate, dextran, gelatin, collagen, and other suitable carriers. In particular embodiments, the organic carrier is sodium hyaluronate. Other possible carriers include glycerin, glycine, glycerol, polyethylene glycol, oils, fatty acids, saccharides, polysaccharides, glycoproteins, and water soluble polymers. In some embodiments, the implants described herein are free of a carrier. In particular embodiments, the implants described herein include a carrier that is free of a lubricant. Alternatively, the implants can include a carrier in an amount less than or equal to about 0.5% by weight, about 1% by weight, about 5% by weight, about 10% by weight, about 15% by weight, about 20% by weight, about 25% by weight, about 30% by weight, about 35% by weight, about 40% by weight, about 45% by weight, about 50% by weight, about 55% by weight, about 60% by weight, about 65% by weight, about 70% by weight, about 75% by weight, about 80% by weight, about 85% by weight, about 90% by weight, or about 95% by weight. In certain embodiments, the implants can include a carrier in an amount of about 10% to about 90% by weight, about 20% to about 85% by weight, about 30% to about 80° A) by weight, or about 50% to about 75% by weight of the implant. Furthermore, in embodiments where the carrier comprises a lubricant, the amount of lubricant in the implant can be in the amounts described above in connection with the amount of carrier in the implant.

In certain embodiments, the implants described herein may include cortical bone units that are supplemented with synthetic material(s) of similar physical dimensions as the cortical bone units. Such synthetic material(s) include, but are not limited to, polymeric hydrogels, biodegradable polymers, rubbers, or other materials that are elastic in nature.

In other embodiments, the implants described herein may include the addition of cells and/or biological or bioactive agents to the cortical bone units, either prior to implantation or post-implantation. Supplementation with cells and/or biological or bioactive agents may induce or accelerate new bone formation within a bony defect following implantation. Such cells may be transplanted cells, and may include, without limitation, autologous cells, allogenic cells, cells derived from bone marrow, e.g., bone marrow aspirate, stem cells, e.g. mesynchemal stem cells, other pluripotent cells, osteoblasts, progenitor cells, chondrocytes, and nucleus pulposus cells. Biological or bioactive agents may include, without limitation, viral particles, plasmids, hormones, extracellular matrix proteins, platelet rich plasma, or growth factors such as those in the TGF-β, FGF, VEGF, IGF, and BMP families.

### Method of Preparing Cortical Bone Units

The cortical bone used to make the cortical bone units can be obtained from long bones. The long bones are first processed into cross-sections of varying thicknesses. In certain embodiments, the cross-sections of cortical bone are at least 0.25 mm thick. In some embodiments, the cross-sections are about 0.25 mm to about 10 mm thick, about 1.0 mm to about 5 mm thick, or about 1.5 mm to about 3 mm thick.

After processing the long bones into cross-sections, the cross-sections of bone are milled into cortical bone units having the desired shape and dimensions. The milling of the bone can be achieved by using a mechanical press, a punching device, a cross-cutting device, or any other art-known device suitable for creating, shaped bone units. Furthermore, the bone can be cleaned before and/or after it is milled. As discussed above, the bone can be cleaned using, for example, hydrogen peroxide or ethanol. The bone can be demineralized before or after milling. Following demineralization, physiological pH levels of the bone can be restored by soaking the at least partially demineralized bone in a buffered salt solution. The cortical bone units can then be lyophilized.

Following lyophilization, the dehydrated, freeze-dried cortical bone units may be re-hydrated using a saline or a buffered salt solution, e.g., phosphate buffered saline (PBS), or a suitable lubricious carrier solution, such as, but not limited to, sodium hyaluronate, such as that discussed above. If a carrier is added, excess carrier solution can removed from the cortical tissue, and the tissue can be loaded into a delivery container that is designed to facilitate minimally invasive delivery of the cortical bone units.

### Implants Comprising Cortical Bone Units

The implants described herein comprise a plurality of the cortical bone units as described herein. They may generally be delivered to and implanted in a cavity that has a volume and is located in the body of a patient for treating the patient, such as repairing defects in bone. Also, as discussed below, the implants are designed to be delivered through a minimally invasive route into a cavity in a patient.

The implants can use used to treat various bones. These bones include without limitation long bones, (e.g., a femur, tibia, fibula, humerus), bones of the spine, pelvic bones, the skull and bones of the extremities. In certain embodiments, as discussed further below, the implants may be used to treat defects of the spine, such as ones in a vertebral body or in an interbody space between two vertebrae.

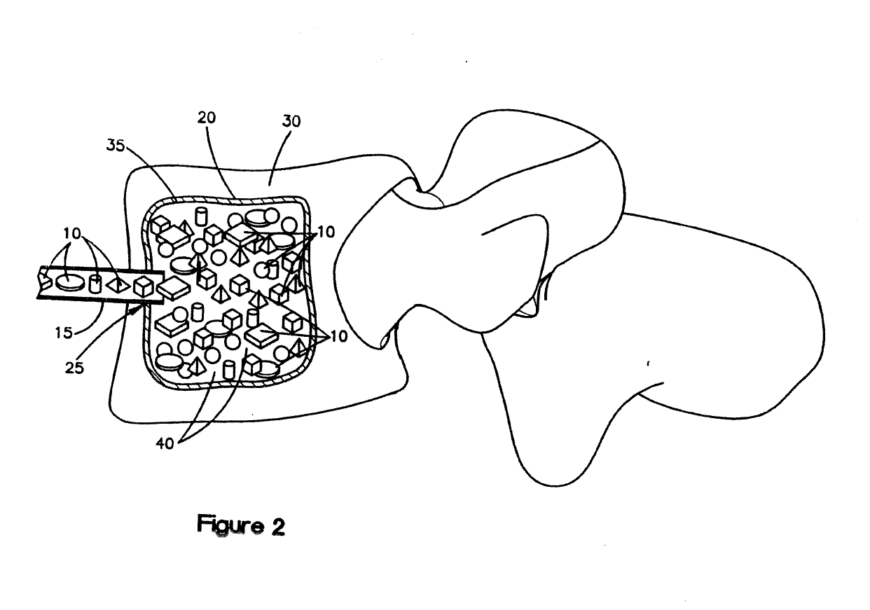

In one embodiment, the implants described herein may be used to repair a fractured or collapsed vertebral body, such as one resulting from a vertebral compression or burst fracture. In some embodiments, the method of treating a vertebral body compression or burst fracture in a patient can comprise the steps of accessing a target vertebral body of the patient, creating a cavity having a volume within the vertebral body, and implanting into the cavity an implant comprising a plurality of separate cortical bone units. FIG. 2 shows an implant being implanted into a vertebral body.

More specifically, first, a target vertebral body 30 in a patient is accessed by positioning a guide wire either into the pedicle or parallel to the pedicle under fluoroscopic guidance. Subsequently, a cannula is placed over the guide wire that serves as an access portal. After the cannula is secured to the vertebral body, the guide wire is removed and cavity creation tools are utilized in order to create space for the implant and/or the implantable container for the implant.

Next, at least one cavity 20 having a volume is created within the target vertebral body 30. Although only one cavity is shown in FIG. 2, in other embodiments, there may be more than one cavity. The cavity 20 has at least one opening 25. The opening 25 of the cavity 20 may be created by, for example, removal of bony vertebral material by, e.g., reaming, drilling, or scraping, followed by evacuation of the bone particles. The cavity may also be enlarged by the expansion of the expandable container under pressurized filling.

After the formation of a cavity 20 in the vertebral body 30, the resulting cavity can be sized, e.g., as described in United States Publication No. 2008/0027546 to Semler et al., which is incorporated herein by reference in its entirety. The sizing step may consist of inserting an inflatable balloon in the cavity and filling the cavity with radio-contrast fluid to a specific pressure between about 30 psi to about 60 psi such that the cavity is visible under fluoroscopy. This step allows visualization of the cavity created and also provides a measurement of the cavity volume, which is used to determine the amount of material needed for the implant.

Next, an implant as described herein, comprising a plurality of separate cortical bone units 10, is inserted into the cavity. As shown in FIG. 2, the cortical bone units 10 can be contained in a delivery container 15, such as that shown in FIG. 1B, for delivery into the cavity 20. In certain embodiments, in order to facilitate delivery, the cortical bone units may be loaded into the delivery container prior to the time of surgery. In alternate embodiments, the cortical bone units may be loaded into the delivery container during the time of surgery. The implants described herein are designed so that it is easy for a surgeon or other assisting persons to load the cortical bone units into such delivery containers.

In some embodiments, as shown in FIG. 2, the delivery container 15 is inserted into the opening 25 of the cavity 20 and the cortical bone units 10 are passed into the cavity 20 located in the vertebral body 30. The cortical bone units 10 are passed into the cavity 20 until the desired amount of cortical bone units 10 is placed into the cavity 20. In some embodiments, the cortical bone units of the implant are inserted directly into the cavity in the patient.

In other embodiments, such as that shown in FIG. 2, an implantable container 35 is inserted into the cavity 20 through the opening 25 before the cortical bone units 10 of the implant are inserted into the cavity 20. The implantable container 35 is initially empty and in a collapsed state such that it can be passed through the opening 25 of the cavity 20. The implant is then inserted into the implantable container 35 that is already located within the cavity 20. After the implant has been implanted, the implantable container 35 and/or cavity 20 may be closed or sealed.

In some embodiments, the implantable container is expandable. The implantable container may be expanded in the cavity before the cortical bone units are inserted therein or expanded by the process of inserting the implant into the container. The implantable container may be made from synthetic materials such as, but not limited to, polyester, or from biological materials such as, but not limited to, allograft bone, dermis, or fascia, hyaluronic acid, collagen, or other structural protein.

In some embodiments, the implantable container is porous and comprises, e.g., a mesh, such as a woven fabric mesh. The implantable container can be a mesh bag. In these configurations, the pores of the implantable container will allow bone to grow into the implant site. The pores of the implantable container may also serve to allow the transfer of fluid and materials, such as cells, between the surrounding tissue and the implant site. Also, the implantable container may have pore sizes that are sufficiently small such that the cortical bone units do not readily fall through the pores. In particular embodiments, the implantable container may also possess radiopaque properties such that it is visible during implantation.

As shown in FIG. 2, when the cortical bone units 10 are implanted into the cavity 20 or implantable container 35 within the cavity 20, there are void spaces 40 between the cortical bone units 10. The void spaces facilitate the transfer of fluid and materials between the surrounding tissue and the implant site, which may facilitate cellular penetration and graft incorporation.

In certain embodiments, the cortical bone units occupy less than 100% of the volume of the cavity or implantable container. For example, the cortical bone units may occupy about 25% to about 99%, about 75% to about 95%, about 75% to about 99% or about 80% to about 90% of the volume of the cavity or implantable container. In some embodiments, the cortical bone units may occupy equal to or greater than about 99%, about 98%, about 97%, about 96%, about 95%, about 94%, about 93%, about 92%, about 91%, about 90%, about 85%, about 80%, about 75%, about 70%, about 65%, about 60%, about 55%, or about 50% of the volume of the cavity or implantable container when implanted therein.

The percentage of the volume of the implantable container occupied by the cortical bone units may be directly related to the size and shape of the cortical bone units. For instance, in certain embodiments wherein the cortical bone units are larger in size, this may create larger void spaces in between each cortical bone unit, leading to a decreased percentage of the volume of the cavity or implantable container occupied by the cortical bone units. Conversely, in certain embodiments wherein the cortical bone units are smaller in size, this may allow for smaller void spaces in between each cortical bone unit, leading to an increased percentage of the volume of the cavity or implantable container occupied by the cortical bone units. Moreover, in certain embodiments wherein the bone units have a certain shape, such as a spherical or cuboidal shape, this may also create larger void spaces in between each cortical bone unit, leading to a decreased percentage of the volume of the cavity or implantable container occupied by the cortical bone units.

In some embodiments, when the implant is implanted in the cavity or implantable container, the packing or bulk density of the cortical bone units in the cavity or implantable container can be about 0.01 g/cc to about 5.00 g/cc, about 0.10 g/cc to about 2.00 g/cc, about 0.20 g/cc to about 1.40 g/cc, about 0.40 g/cc to about 1.00 g/cc, about 0.50 g/cc to about 0.80 glee, or about 0.50 g/cc to about 1.00 g/cc based on dry weight of the bone. In particular embodiments, the implants described herein have a packing density of about 0.50 g/cc to about 0.80 g/cc based on dry weight of bone. In other embodiments, the implants described herein have a packing density of about 0.60 g/cc to about 0.80 g/cc based on dry weight of the bone units or implant material.

The amounts of cortical bone units that are implanted into the implant site may be varied for the specific size of the cavity. In certain embodiments, the volume of the implant comprising the cortical bone units can be greater than that of the initial volume of the cavity. In such embodiments, the implant may provide a degree of restoration of vertebral body shape or height in a collapsed or fractured vertebral body. The implants described herein may also possess mechanical properties that withstand the compressive loads in the spine when implanted into the cavity of the patient.

After the cortical bone units are inserted into the cavity or implantable container within the cavity, the opening of the cavity and/or implantable container may be left open. Alternatively, after the implant is implanted into the cavity, the opening to the cavity and/or implantable container may be sealed with a material including, but not limited to a biocompatible sealant. Materials that may be used as biocompatible sealants include, without limitation, an allograft bone plug, a ceramic, polymeric or metallic plug, and fibrin glue.

Moreover, in certain embodiments, the implants can be used to treat spinal discs located between adjacent vertebrae. In some embodiments, the implant can be used to create a fusion between two adjacent vertebral bodies. This type of procedure can be used to address conditions associated with mild to severe disc degeneration or other spinal deformities. In one embodiment, the fusion procedure may comprise forming at least one cavity, having a volume and an opening, between two adjacent vertebral bodies. An implant comprising a plurality of the cortical bone units described therein can then be implanted into the cavity.

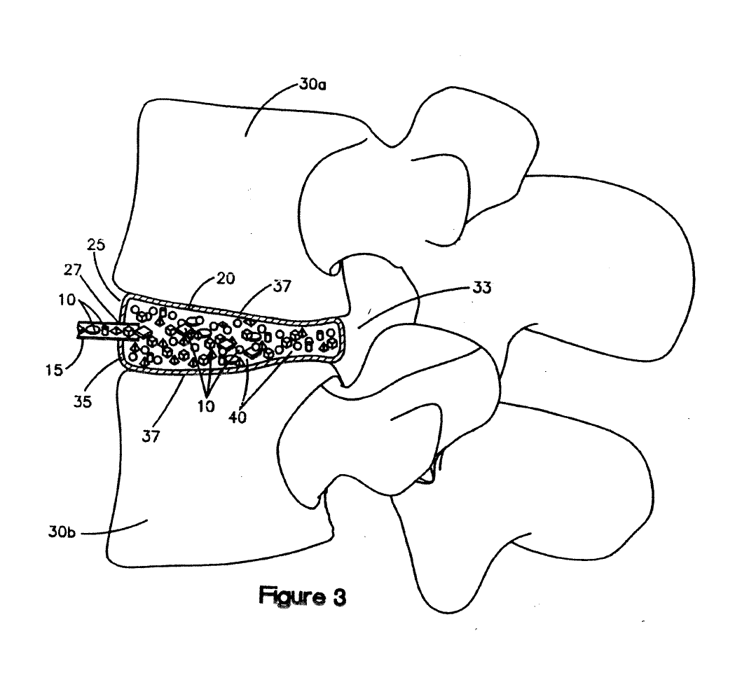

FIG. 3 shows an implant being implanted in the space between two vertebral bodies. In this embodiment, a targeted intervertebral disc space 33 is accessed. The disc space in a patient can be accessed by positioning a guide wire either into the disc from either an anterior, posterior, posterolateral, anterolateral, or lateral approach to the spine under fluoroscopic guidance. Subsequently, a cannula is placed over the guide wire that serves as an access portal. After the cannula is secured to the disc, the guide wire is removed and cavity creation tools are utilized in order to create space for the implant and/or the expandable container for the implant.

Thereafter, all or a portion of the intervertebral disc is removed to create a cavity 20, having a volume, between the two adjacent intervertebral bodies 30a and 30b. The cavity may be created by removing at least a portion of the intervertebral disc. e.g., by microdiscectomy, minimally invasive nucleotomy, or by, e.g., reaming, drilling, gouging or scraping followed by evacuation of the disc fragments An opening to the cavity may be created during the formation of the cavity as described herein.

After the cavity is created, the endplates 37 of the vertebral bodies 30a and 30b can be decorticated to access bleeding bone. The endplates 37 can be decorticated by gouging, scraping, cutting or piercing tools. Also, the cavity can be sized before the implant is implanted by, for example, the methods discussed above.

An implantable container 35, such as that discussed herein, can be used. As shown in FIG. 3, an implantable container 35 is inserted into the cavity 20 before the cortical bone units 10 of the implant are inserted into the cavity 20. The implantable container 35 may expanded before the implant is placed into the implantable container. A delivery container 15 is inserted into the opening 27 of the implantable container 35 and the cortical bone units 10 are passed into the implantable container 35 in the cavity 20. If an implantable container is not used, a delivery container 15 can be inserted into the opening of the cavity 25 and the cortical bone units 10 can be passed into the cavity 20. The cortical bone units 10 are passed into the container 35 or cavity 20 until the desired amount of cortical bone units 10 is placed into the implantable container 35 or cavity 20. After the implant has been implanted, the implantable container 35 and/or cavity 20 may be closed or sealed.

As illustrated in FIG. 3, when the cortical bone units 10 are implanted into the cavity 20 between the vertebral bodies 30a and 30b or implantable container 35 within the cavity 20, there are void spaces 40 between the cortical bone units 10. As discussed above, the void spaces facilitate the transfer of fluid and materials between the surrounding tissue and the implant site, which may facilitate cellular penetration and graft incorporation.

In addition, in certain embodiments, as described above, the cortical bone units may occupy a certain percentage of the volume of the cavity or implantable container. Also, in some embodiments, when the implant is implanted in the cavity or implantable container, the packing or bulk density of the cortical bone units can be a certain value.

As with the implants used to address vertebral fractures discussed previously, the amount of cortical bone units that is implanted into the implant site may be varied. For instance, the volume of the implant comprising the cortical bone units can be greater than that of the initial volume of the cavity so that the implant may provide mechanical properties that withstand the compressive loads in the spine when implanted into the cavity of the patient.

Additionally, in certain embodiments, the implants described herein may be used to repair or replace a part or all of a spinal disc without fusion of the vertebrae. Also, the implant may be used to augment the spinal disc or restore the height of the spinal disc. For example, the implant may be used to replace all or part of the nucleus pulposus of the spinal disc. In these embodiments, an opening is made in the spinal disc. All or part of the nucleus pulposus is removed to create a cavity in the spinal disc that is located between two adjacent vertebrae. The methods described above in creating a cavity for spinal fusion may be used to remove the nucleus pulposus and create the cavity in the spinal disc. The implant is then inserted into the cavity in the spinal disc. Moreover, an implantable container may be used as described above. The methods described for inserting the implant and implantable container in connection with spinal fusions can be used to insert the implant and implantable container into the cavity in the spinal disc.

Furthermore, in certain embodiments where the implants described herein are used to repair or replace a part of a spinal disc without fusion of the vertebrae, at least some or all of the cortical bone units can be non-osteoinductive. The cortical bone units can be rendered non-osteoinductive by, for example, exposing the cortical bone to hydrogen peroxide for a certain amount of time during the preparation of the bone used in the implants. In one embodiment, after the cortical bone is demineralized, it can be exposed to hydrogen peroxide for at least 1 hour. In other embodiments, the cortical bone can be rendered non-osteoinductive by exposing the cortical bone to heat, radiation or chemicals.

The description contained herein is for purposes of illustration and not for purposes of limitation. The methods and constructs described herein can comprise any feature described herein either alone or in combination with any other feature(s) described herein. Changes and modifications may be made to the embodiments of the description. Furthermore, obvious changes, modifications or variations will occur to those skilled in the art. Also, all references cited above are incorporated herein, in their entirety, for all purposes related to this disclosure.

The following illustrative examples are set forth to assist in understanding the methods and constructs described herein and do not limit the claimed methods and constructs.

### EXAMPLES

### Example 1

### Preparation of Demineralized Cortical Bone Units

Fully demineralized cortical bone units were processed by cutting a long bone shaft into 2.4 mm thick cortical rings using a band saw. Lipids were then removed from the cortical rings using Tween 80 solution, cleaned using hydrogen peroxide, and then demineralized with an extended soak using 0.6N HCl to reach a residual calcium level below 0.5 wt %. The demineralized cortical rings were then cut into cubes with 2.4 mm sides. Afterwards, the pH was restored to physiological levels using a buffered salt solution. The demineralized cortical bone units was then soaked in ethanol, rinsed with water, and then lyophilized to a residual moisture content of less than 6 wt %.

### Example 2

### Comparative Example—Preparation of a Composition of Non-Demineralized Corticocancellous Bone Granules, Demineralized Cortical Bone Powder and Sodium Hyaluronate

A mixture containing non-demineralized corticocancellous bone granules, demineralized cortical bone powder and sodium hyaluronate was produced as follows. Pieces of cortical and cancellous bone were cut into smaller pieces and delipidized using a surfactant solution. Subsequently, the cortical bone pieces and then the cancellous bone pieces were separately milled into granules with a size range of 212 μm to 850 μm. The cortical bone granules were then divided into two portions. The first portion was combined with cancellous granules in an 80:20 cortical to cancellous ratio by weight and then further cleaned with peroxide and ethanol. Following this step, the non-demineralized corticocancellous granules were lyophilized to a residual moisture content of less than 6 wt %. The second portion of cortical bone granules was used to make demineralized cortical bone powder or demineralized bone matrix (DBM). The second portion of cortical bone granules was milled into powder. The cortical bone powder was soaked in peroxide and ethanol and then demineralized using 0.6N HCl to reach a residual calcium level below 0.5 wt %. The DBM was then lyophilized to a residual moisture content of less than 6 wt %. Subsequently, the final composition was obtained by mixing together 4 parts sodium hyaluronate, 4 parts of the non-demineralized corticocanellous granules, and 1 part of the DBM.

### Example 3

### Comparative Packing Densities Required to Sustain Loads in Confined Compression

Sustained loading testing was performed to determine the comparative material packing or bulk densities of (1) the demineralized cortical bone units prepared as described in Example 1 above (“the test samples”), and (2) the composition prepared as described in Example 2 above (“the control samples”), which was a mixture of non-demineralized corticocancellous granules, DBM and sodium hyaluronate. A Bionix 858 Test System (MTS, Minneapolis, Minn.) was used to determine the packing densities.

For the test samples, aliquots of the lyophilized demineralized cortical bone units of Example 1 (0.6 g dry weight each) were re-hydrated in excess saline and then loaded into a porous confined compression chamber. The demineralized cortical bone units were in the shaped of cubes, in which each side was about 2.4 mm. The chamber was 12.5 mm in diameter and contained equally spaced 1 mm pores around its circumference to allow for fluid exchange between the chamber and a surrounding saline bath. A custom piston (12.3 mm in diameter) was fabricated to provide compression to the test sample inside the chamber. Prior to testing, the saline bath was filled with sufficient saline in order to cover the pores of the compression chamber.

For the control samples, approximately 1 cc of the formulation of Example 2 was added into the confined compression chamber prior to testing. After testing, the control samples in their entirety were carefully collected and lyophilized in order to determine the dry weight of material.

During the test procedure, the samples were first preconditioned by cycling the piston up and down so that it applies pressure between 20 psi and 150 psi 100 for cycles at 0.5 hz. Following preconditioning, the piston applied constant pressure to the samples for ten minute intervals at four increasing fixed pressures in a stepwise process. In this manner, the samples were subjected to 20, 50, 150, and 400 psi in order to cover a wide range of physiologically relevant loading levels for the spine. The height of each sample was recorded during the testing at each applied pressure with a data sampling frequency of 10 Hz.

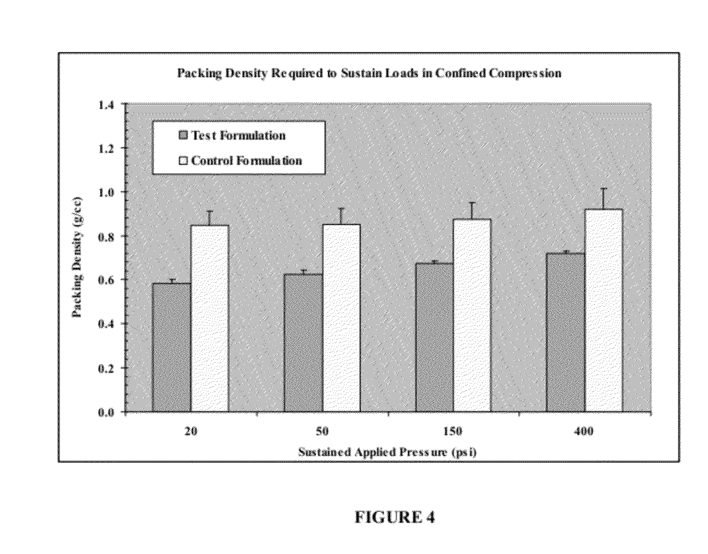

Following the completion of the test, the data was analyzed by determining the average of the last ten height values that were recorded for each sample at each fixed pressure. The packing density was then calculated by using this value along with the original dry weight of the samples and the dimensions of the confined compression chamber. FIG. 4 represents the average of multiple test and control samples of each condition, and the error bars reflect one standard deviation from the mean.

As can be seen from FIG. 4, for each of the sustained applied pressures, the packing density of the test samples or “Test Formulation” ranged from about 0.5 g/cc to about 0.8 g/cc based on dry weight. For each of the sustained applied pressures, the packing density of the control samples or “Control Formulation” ranged from about 0.8 g/cc to about 1.0 g/cc based on dry weight. As shown in FIG. 4, the test samples were able to sustain the same amount of pressure with a lower packing density than the control samples at each level of applied pressure tested.

### Example 4

### Study of Bone Compositions Implanted into Sheep

An animal study involving sheep was performed to evaluate (A) two compositions comprising demineralized sheep cortical bone units (“Test Compositions 1 and 2”) and (B) a composition comprising non-demineralized corticocanellous granules, demineralized cortical bone powder and sodium hyaluronate (“Control Composition”). The compositions were implanted into vertebral bodies of the sheep's spines using a sheep vertebral bone void model.

Test Composition 1 was comprised of demineralized sheep cortical bone units, which were in the shape of cubes having sides of about 2.4 mm, and sodium hyaluronate (HY). Test Composition 2 was comprised of demineralized sheep cortical bone units, which were in the shape of cubes having sides of about 2.4 mm, and a phosphate buffered saline solution (PBS). The demineralized sheep cortical bone units of Test Compositions 1 and 2 were prepared in a manner similar to that described in Example 1 above. The Control Composition was comprised of non-demineralized sheep corticocancellous granules, demineralized sheep cortical bone powder and sodium hyaluronate. The Control Composition was prepared in a manner similar to that described in Example 2 above. Test Compositions 1 and 2 as well as the Control Composition were packaged in small diameter, stainless steel tubes for convenient delivery of the compositions into the implantation site.

Vertebral body augmentation procedures were performed on thirteen skeletally mature sheep of approximately equal size. The procedures were performed under general anesthesia and under sterile conditions. For each sheep, a lateral retroperitoneal approach to three vertebral bodies, (L3, L4, and L5), was made. A standard-sized 8 mm diameter by 15 mm deep hole was drilled into each of the three vertebral bodies in the sheep. A small amount of bone was removed from each of the vertebral bodies to create voids. Each void was either left empty or filled with Test Composition 1, Test Composition 2 or the Control Composition. The voids that were left empty were used as a negative control. Animals were sacrificed at either 6 or 12 weeks. The vertebral bodies were harvested and CT scanned to obtain radiographs thereof. Furthermore, the vertebral bodies were labeled, fixed in 70% ethanol and subsequently prepared for histology evaluations.

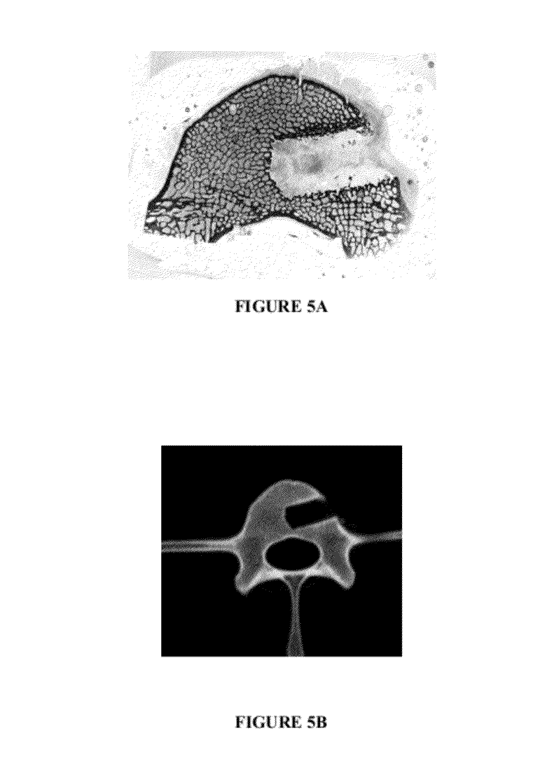

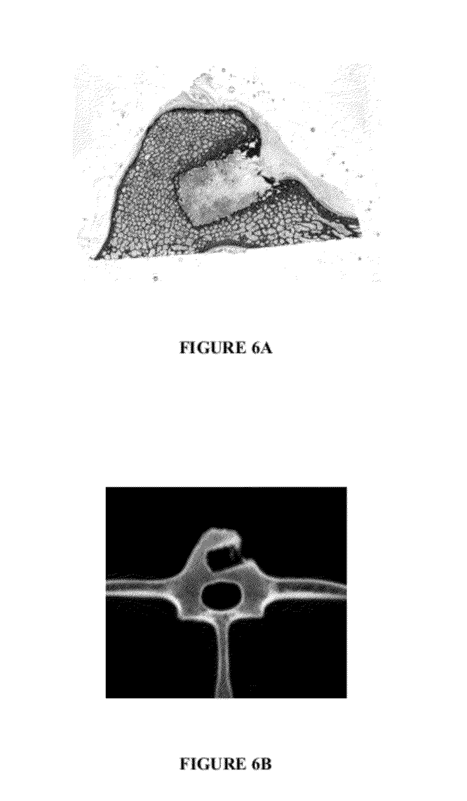

The voids in the vertebral bodies that had been left empty showed little or no new bone formation, with many being filled with fibrous tissue and fat. FIGS. 5A and 6A show histology samples of voids in vertebral bodies that were left empty at 6 weeks and 12 weeks, respectively, after the voids were formed. As shown in FIGS. 5A and 6A, there was a lack of bone formation throughout the voids. FIGS. 5B and 6B are radiographs, (obtained by CT imaging), of the voids of the vertebral bodies shown in FIGS. 5A and 6A respectively. The radiolucent areas inside the voids show that lack of new bone growth.

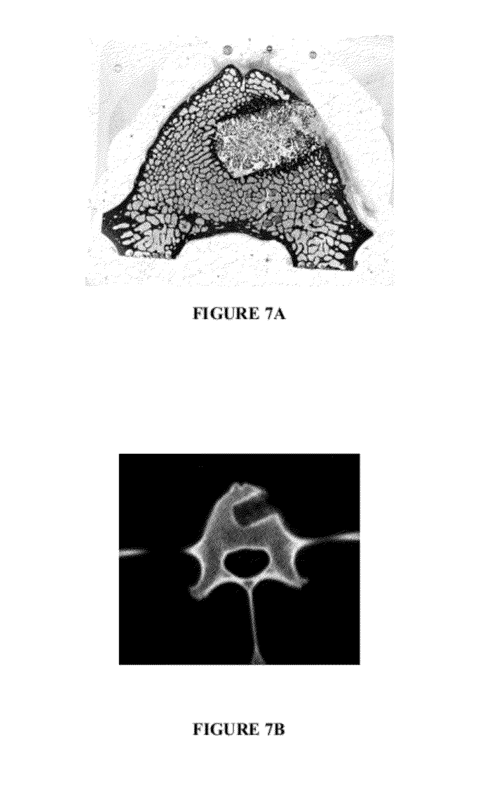

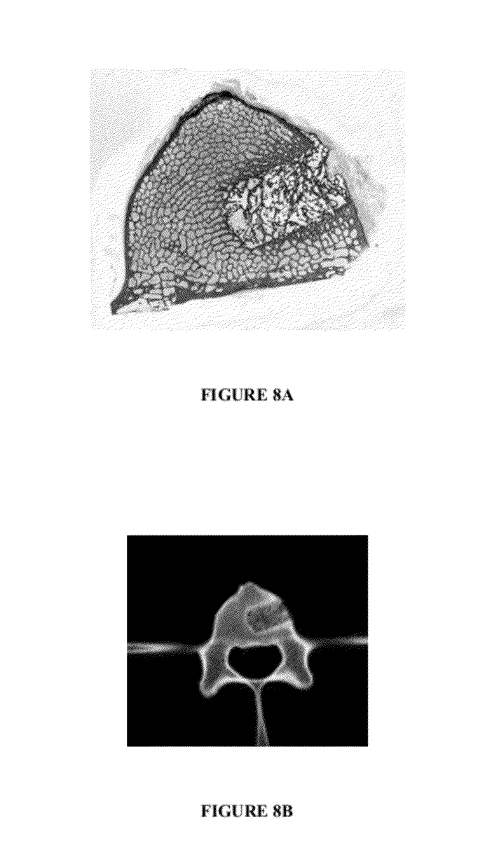

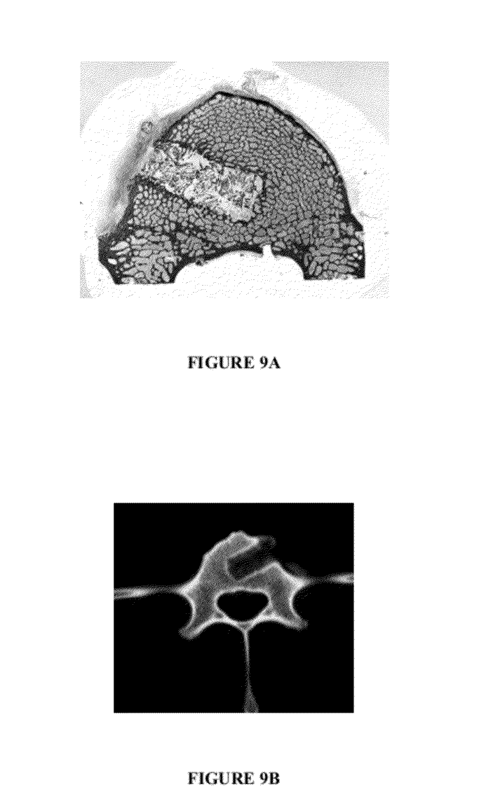

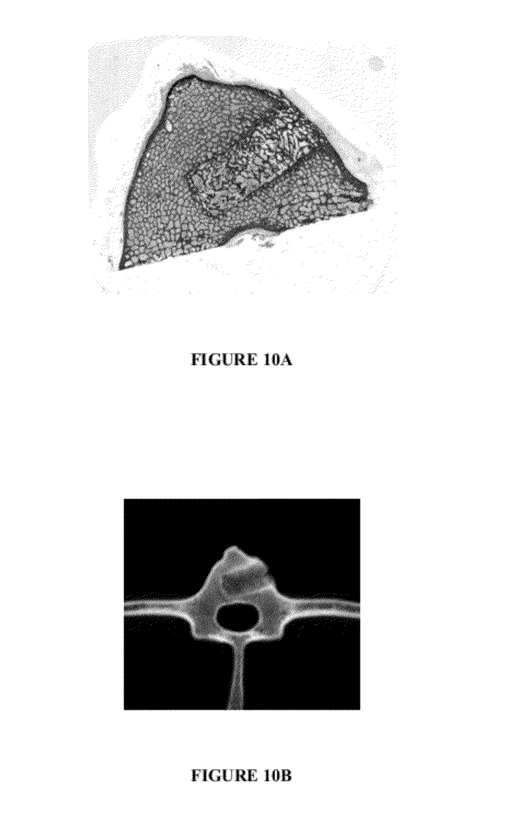

Histological results from the study showed that the voids in the vertebral bodies, which were filled with either Test Compositions 1 or 2, contained much more newly formed bone at 6 weeks and at 12 weeks after the voids were formed and filled than the empty voids. FIG. 7A shows a void of a vertebral body at 6 weeks after the void was filled with Test Composition 1 (demineralized cortical bone units and HY). This figure shows the remodeling of the demineralized cortical bone units of Test Composition 1 as new bone formation is beginning to occur throughout the void. FIG. 8A shows a void of a vertebral body at 12 weeks after the void was filled with Test Composition 1. As shown in the figure, at 12 weeks, there was new mineralized bone throughout the void. FIG. 9A shows a void of a vertebral body at 6 weeks after the void was filled with Test Composition 2 (demineralized cortical bone units and PBS). This figure shows the remodeling of the demineralized cortical bone units of Test Composition 2 as new bone formation is beginning to occur throughout the void. FIG. 10A is a histology sample of a void of a vertebral body at 12 weeks after the void was filled with Test Composition 2. The histology sample in FIG. 10A shows the formation of new mineralized bone throughout the defect.

Radiographs obtained by CT imaging showed that by 12 weeks, new mineralized bone had formed in voids filled with Test Compositions 1 and 2. At 6 weeks, the radiographs of the voids in the vertebral bodies that were filled with Test Compositions 1 and 2 were mostly radiolucent since new mineralized bone had not yet formed. By 12 weeks, radiographs of the voids that were filled with Test Compositions 1 and 2 were more opaque than similar voids at 6 weeks and also more radiopaque than the empty control voids, which appeared radiolucent at both 6 and 12 weeks. FIGS. 7B and 8B are radiographs, obtained from CT imaging, of the voids of the vertebral bodies shown in FIGS. 7A and 8A, respectively, that were tilled with Test Composition 1. The radiograph in FIG. 7B shows a radiolucent area inside the void as new mineralized bone has yet to form by 6 weeks. In FIG. 8B, the void is radiopaque at 12 weeks, indicating the formation of new mineralized bone in the void. FIGS. 9B and 10B are radiographs, obtained from CT imaging, of the voids of the vertebral bodies shown in FIGS. 9A and 10A, respectively, that were filled with Test Composition 2. The radiograph of FIG. 9B shows a radiolucent area inside the void since new mineralized bone has not yet formed by 6 weeks. FIG. 10B shows a void that is radiopaque at 12 weeks, which indicates that new mineralized bone has formed.

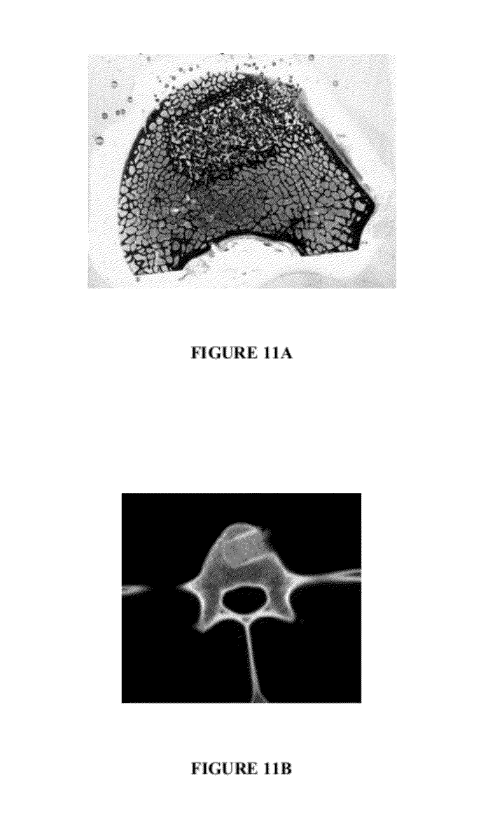

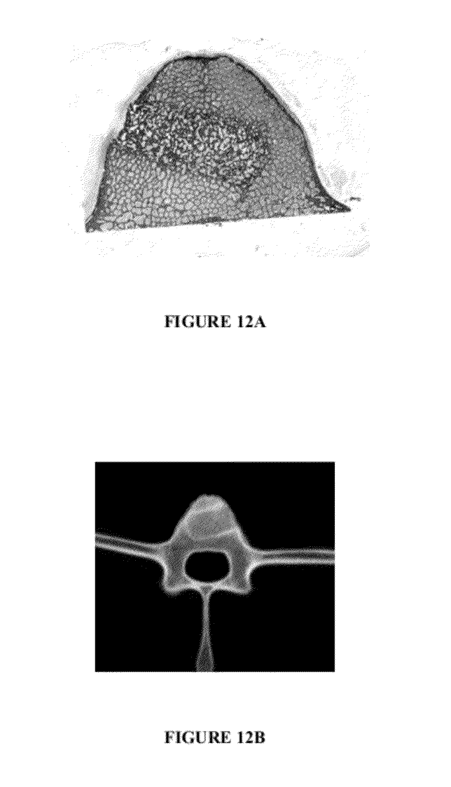

The histology results showed that voids that were filled with the Control Composition appeared to be filled with mineralized bone at both 6 and at 12 weeks. But at 6 weeks, a large portion of this mineralized bone appeared to be the non-demineralized corticocancellous granules that was originally present in the Control Composition. FIG. 11A shows a histology sample of a void of a vertebral body that was filled with the Control Composition at 6 weeks after the void was filled. This sample indicated the presence in the void of the non-demineralized corticocancellous granules that were present in the Control Composition. FIG. 12A shows a histology sample of a void of a vertebral body that had been filled with the Control Composition for 12 weeks. This figure shows the presence of newly remodeled woven bone in the void.

Radiographs of the voids tilled with the Control Composition show that these voids appeared radiopaque at both 6 and 12 weeks. FIG. 11B is a radiograph obtained by CT imaging of a void of a vertebral body filled with the Control Composition at 6 weeks. There is a radiopaque signal inside the void that is the result of the non-demineralized bone in the Control Composition. FIG. 12B is a radiograph of a void of a vertebral body that had been tilled with the Control Composition for 12 weeks. This radiograph shows a radiopaque signal inside the void that is similar to the one seen at 6 weeks in FIG. 11B. Given this observation, it was more apparent from the radiographs that newly formed bone was present in the voids filled with Test Formulations 1 or 2 versus the radiographs of the voids tilled by the Control Composition that contained non-demineralized corticocancellous granules.

What is claimed is:

1. An implant comprising a plurality of separate cortical bone units that are at least partially demineralized and osteoinductive,
wherein the cortical bone units have at least one dimension greater than about 1.0 mm, and
wherein when the cortical bone units are implanted into a cavity that has a volume, there are void spaces between the cortical bone units in the cavity.

2. The implant of claim 1, wherein the cavity is located in a patient.

3. The implant of claim 1, wherein the cortical bone units are implanted into an implantable container located within the cavity.

4. The implant of claim 1, wherein at least one of the cortical bone units has a cylindrical, spherical, pyramidal, ovoid, discoid, oblong, or cuboidal shape.

5. The implant of claim 1, wherein the cortical bone units have at least one dimension from about 1.5 mm to about 5.0 mm.

6. The implant of claim 1, wherein the implant includes less than or equal to about 5% by weight of cortical bone units having at least one dimension less than about 1.0 mm.

7. The implant of claim 1, wherein the cortical bone units are formed from human bone.

8. The implant of claim 1, wherein the implant includes less than or equal to about 1% by weight of cancellous bone.

9. The implant of claim 1, wherein the cortical bone units are fully demineralized.

10. The implant of claim 1, wherein the implant includes less than or equal to about 1% by weight of non-demineralized bone.

11. The implant of claim 1, further comprising a radiopaque marker.

12. The implant of claim 1, further comprising a carrier, wherein the carrier comprises saline, sodium hyaluronate or hyaluronic acid, and wherein the carrier is mixed with the cortical bone units.

13. The implant of claim 1, wherein the implant includes less than or equal to about 1% by weight of a carrier.

14. The implant of claim 3, wherein the cortical bone units occupy about 75% to about 99% of the volume of the cavity or implantable container.

15. The implant of claim 1, wherein when the implant is implanted in the cavity, the packing density of the cortical bone units in the cavity is of about 0.5 g/cc to about 1.0 glee based on dry weight of the cortical bone units.

16. A method of treating bone comprising:
(a) forming at least one cavity, having a volume and at least one opening, within the bone; and
(b) implanting into the cavity an implant comprising a plurality of separate cortical hone units that are at least partially demineralized and osteoinductive,
wherein the cortical bone units have at least one dimension greater than about 1.0 mm, and
wherein after the implant has been implanted in the cavity there are void spaces between the cortical bone units in the cavity.

17. The method of claim 16, further comprising the step of sealing the opening of the cavity after the implant has been implanted in the cavity.

18. The method of claim 16, further comprising the step of inserting an implantable container into the cavity prior to implanting the implant into the cavity so that when the implant is implanted into the cavity, the implant will be contained in the implantable container.

19. The method of claim 18, wherein the implantable container is a mesh bag.

20. The method of claim 16, wherein prior to implanting the implant in the cavity, the plurality of cortical bone units are contained in a delivery container which comprises a cannula, syringe, cartridge, hollow rod, hollow delivery tube, or fill tube.

21. The method of claim 16, wherein at least one of the cortical bone units has a cylindrical, spherical, pyramidal, ovoid, discoid, oblong or cuboidal shape.

22. The method of claim 16 wherein at least one of the cortical bone units have at least one dimension from about 1.5 mm to about 5.0 mm.

23. The method of claim 16, wherein the implant includes less than or equal to about 5% by weight of cortical bone units having at least one dimension less than about 1.0 mm.

24. The method of claim 16, wherein the cortical bone units are formed from human hone.

25. The method of claim 16, wherein the implant includes less than or equal to about 1% by weight of cancellous bone.

26. The method of claim 16, wherein the cortical bone units are fully demineralized.

27. The method of claim 16, wherein the implant includes less than or equal to about 1% by weight of non-demineralized bone.

28. The method of claim 16, further comprising a radiopaque marker.

29. The method of claim 16, further comprising a carrier, wherein the carrier comprises saline, sodium hyaluronate or hyaluronic acid, and wherein the carrier is mixed with the cortical bone units.

30. The method of claim 16, wherein the implant includes less than or equal to about 1% by weight of a carrier.

31. The method of claim 18, wherein the cortical bone units occupy about 75% to about 99% of the volume of the cavity or implantable container.

32. The method of claim 16, wherein when the implant is implanted into the cavity, the packing density of the cortical bone units in the cavity is of about 0.5 g/cc to about 1.0 g/cc based on dry weight of the cortical bone units.

33. A method of treating a vertebral body in a patient comprising:
(a) forming at least one cavity, having a volume and at least one opening, within the vertebral body; and
(b) implanting into the cavity an implant comprising a plurality of separate cortical bone units that are at least partially demineralized and osteoinductive,
wherein the cortical bone units have at least one dimension greater than about 1.0 mm, and
wherein after the implant has been implanted in the cavity there are void spaces between the cortical bone units in the cavity.

34. The method of claim 33, wherein the vertebral body is fractured and the method is for treating the fractured vertebral body.

35. The method of claim 33, further comprising the step of sealing the opening of the cavity after the implant has been implanted in the cavity.

36. The method of claim 33, further comprising the step of inserting an implantable container into the cavity prior to implanting the implant into the cavity so that when the implant is implanted into the cavity, the implant will be contained in the implantable container.

37. The method of claim 36, wherein the implantable container is a mesh bag.

38. The method of claim 33, wherein prior to implanting the implant in the cavity, the plurality of cortical bone units are contained in a delivery container which comprises a cannula, syringe, cartridge, hollow rod, hollow delivery tube, or fill tube.

39. The method of claim 33, wherein at least one of the cortical bone units has a cylindrical, spherical, pyramidal, ovoid, discoid, oblong or cuboidal shape.

40. The method of claim 33, wherein at least one of the cortical bone units have at least one dimension from about 1.5 mm to about 5.0 mm.

41. The method of claim 33, wherein the implant includes less than or equal to about 5% by weight of cortical bone units having at least one dimension less than about 1.0 mm.

42. The method of claim 33, wherein the cortical bone units are formed from human bone.

43. The method of claim 33, wherein the implant includes less than or equal to about 1% by weight of cancellous bone.

44. The method of claim 33, wherein the cortical bone units are fully demineralized.

45. The method of claim 33, wherein the implant includes less than or equal to about 1% by weight of non-demineralized bone.

46. The method of claim 33, further comprising a radiopaque marker.

47. The method of claim 33, further comprising a carrier, wherein the carrier comprises saline, sodium hyaluronate or hyaluronic acid, and wherein the carrier is mixed with the cortical bone units.

48. The method of claim 33, wherein the implant includes less than or equal to about 1% by weight of a carrier.

49. The method of claim 36, wherein the cortical bone units occupy about 75% to about 99% of the volume of the cavity or implantable container.

50. The method of claim 33, wherein when the implant is implanted into the cavity, the packing density of the cortical bone units in the cavity is of about 0.5 g/cc to about 1.0 g/cc based on dry weight of the cortical bone units.

51. A method of treating a spinal disc in a patient comprising:
(a) forming at least one cavity, having a volume and at least one opening, herein the cavity is located between two adjacent vertebral bodies; and
(b) implanting into the cavity an implant comprising a plurality of separate cortical bone units that are at least partially demineralized and osteoinductive,
wherein the cortical bone units have at least one dimension greater than about 1.0 mm, and
wherein after the implant has been implanted in the cavity there are void spaces between the cortical bone units in the cavity.

52. The method of claim 51, wherein the spinal disc is degenerated and the method is for treating the degenerated spinal disc.

53. The method of claim 51, wherein the implant is used to create a fusion between the two adjacent vertebral bodies.

54. The method of claim 51, wherein at least one of the vertebral bodies has an endplate and the method further comprises decorticating the endplate prior to implanting the implant into the cavity.

55. The method of claim 51, wherein the cavity is located within the spinal disc.

56. The method of claim 51, further comprising the step of sealing the opening of the cavity after the implant has been implanted into the cavity.

57. The method of claim 51, further comprising the step of inserting an implantable container into the cavity prior to implanting the implant into the cavity so that when the implant is implanted into the cavity, the implant will be contained in the implantable container.

58. The method of claim 57, wherein the implantable container is a mesh bag.

59. The method of claim 51, wherein prior to insertion of the implant in the cavity, the plurality of cortical bone units are contained in a delivery container which comprises a cannula, syringe, cartridge, hollow rod, hollow delivery tube, or fill tube.

60. The method of claim 51, wherein at least one of the cortical bone units has a cylindrical, spherical, pyramidal, ovoid, discoid, oblong, or cuboidal shape.

61. The method of claim 51, wherein the cortical bone units have at least one dimension from about 1.5 mm to about 5.0 mm.

62. The method of claim 51, wherein the implant includes less than or equal to about 5% by weight of cortical bone units having at least one dimension less than about 1.0 mm.

63. The method of claim 51, wherein the cortical bone units are formed from human bone.

64. The method of claim 51, wherein the implant includes less than or equal to about 1% by weight of cancellous bone.

65. The method of claim 51, wherein the cortical bone units are fully demineralized.

66. The method of claim 51, wherein the implant includes less than or equal to about 1% by weight of non-demineralized bone.

67. The method of claim 51, further comprising a radiopaque marker.

68. The method of claim 51, further comprising a carrier, wherein the carrier comprises saline, sodium hyaluronate or hyaluronic acid, and wherein the carrier is mixed with the cortical bone units.

69. The method of claim 51, wherein the implant includes less than or equal to about 1% by weight of a carrier.

70. The method of claim 57, wherein the cortical bone units occupy about 75% to about 99% of the volume of the cavity or implantable container.

71. The method of claim 51, wherein when the implant is implanted into the cavity, the packing density of the cortical bone units in the cavity is of about 0.5 g/cc to about 1.0 g/cc based on dry weight of the cortical bone units.

72. A method of treating a spinal disc in a patient comprising:
(a) forming at least one cavity, having a volume and at least one opening, wherein the cavity is located within the spinal disc; and
(b) implanting into the cavity an implant comprising a plurality of separate cortical bone units that are at least partially demineralized and non-osteoinductive,
wherein the cortical bone units have at least one dimension greater than about 1.0 mm, and
wherein after the implant has been implanted in the cavity there are void spaces between the cortical bone units in the cavity.

73. The method of claim 72, further comprising the step of inserting an implantable container into the cavity prior to implanting the implant into the cavity so that when the implant is implanted into the cavity, the implant will be contained in the implantable container

74. The method of claim 72, wherein the cortical bone units have at least one dimension from about 1.5 mm to about 5.0 mm.

75. The method of claim 72, wherein the implant includes less than or equal to about 5% by weight of cortical bone units having at least one dimension less than about 1.0 mM.

76. The method of claim 73 wherein the cortical bone units are formed from human bone.

77. The method of claim 72, wherein the cortical bone units are fully demineralized.

78. The method of claim 72, wherein the implant includes less than or equal to about 1% by weight of non-demineralized bone.

79. The method of claim 73, wherein the cortical bone units occupy about 75% to about 99% of the volume of the cavity or implantable container.

80. The method of claim 72, wherein when the implant is implanted into the cavity, the packing density of the cortical bone units in the cavity is of about 0.5 g/cc to about 1.0 g/cc based on dry weight of the cortical bone units.
